(19) United States
(12) Patent Application Publication
(10) Pub. No.: US 2014/0283717 A1
(43) Pub. Date: Sep. 25, 2014

(54) METHOD FOR CONVERTING ORGANIC MATERIAL INTO A RENEWABLE FUEL

(71) Applicant: SGC Advisors, LLC, Atlanta, GA (US)

(72) Inventors: Kevin M. Bolin, Atlanta, GA (US); James R. Miller, San Juan Capistrano, CA (US)

(73) Assignee: SGC Advisors, LLC, Atlanta, GA (US)

(21) Appl. No.: 14/296,397

(22) Filed: Jun. 4, 2014

Related U.S. Application Data
(63) Continuation of application No. 12/755,675, filed on Apr. 7, 2010, now abandoned.
(60) Provisional application No. 61/167,207, filed on Apr. 7, 2009.

(51) Int. Cl. C10L 5/46; F23G 5/04; B01D 3/06; C12P 5/02; B01F 15/06

(52) U.S. Cl. CPC C10L 5/46; C12P 5/023; B01F 15/06; B01D 3/06; F23G 5/04
USPC 110/221; 44/605; 435/167; 366/144; 203/88; 203/77

(57) ABSTRACT

Processes for converting organic material into renewable fuel products. A feedstock containing organic material is processed at an elevated pressure and temperature to lyse, decarboxylate, and carbonize cell structures. A portion of the processed slurry may be recirculated and mixed with cool, pressurized feedstock prior to reaching a mechanical mixing device to preheat and reduce the viscosity of the feedstock. The pressure and temperature are reduced, which may occur simultaneously to flash volatile materials, such as ammonia, out of the slurry, thereby reducing the presence of the materials in the final product and allowing recovery of the materials. The processed slurry may be treated with a halide to reduce mercury emissions in the final product. The treated slurry is mechanically and thermally dewatered resulting in a renewable fuel product in dried particulate or pelletized form that is a viable energy source having a positive heating value.

### CROSS-REFERENCE TO RELATED APPLICATIONS

This application is a continuation of U.S. patent application Ser. No. 12/755,675, filed Apr. 7, 2010, which claims priority from U.S. Provisional Patent Application Ser. No. 61/167,207, filed Apr. 7, 2009, both entitled “METHOD FOR CONVERTING ORGANIC MATERIAL INTO A RENEWABLE FUEL,” the disclosures of which are hereby expressly incorporated by reference herein in their entirety.

### FIELD

The present invention relates to the processing of organic material and, in an embodiment, relates to processes for converting organic material into renewable fuel products.

### BACKGROUND

Organic material, such as sludge from sewage and wastewater treatment plants (WWTP), represents a serious disposal problem. This sludge generally contains a mixture of solids, commonly referred to as biosolids, and varying amounts of free water.

The large volume of cell-bound water in biosolids makes the disposal of sewage sludge containing biosolids challenging. In particular, the cost of incinerating sewage sludge is prohibitive because the cell-bound water gives biosolids a net negative lower heating value. Similarly, if sewage sludge is thermally dewatered, the process may have a net negative energy balance due to the energy required to evaporate water from the sewage sludge. Also, the cost of transporting sewage sludge is significant because the cell-bound water impacts the weight of the sludge. Usually the WWTP must pay a “tipping fee” to have another party dispose of the biosolids, and sludge containing biosolids is presently landfilled, land-applied, or dried and used as a fertilizer. However, these disposal methods may have negative environmental effects, such as the generation of undesirable odors and the contamination of soil or groundwater by living disease-causing organisms, toxic heavy metals, and/or other chemical or pharmaceutical compounds contained in the biosolids. Between approximately 7.1 and 7.6 million dry (short) tons of biosolids are produced each year in the U.S. alone, thus, an adequate disposal method is important.

In addition to the current need for an adequate method of disposing of biosolids, there is growing public support for increased utilization of renewable, or “green”, energy sources. Well-known forms of renewable energy include solar energy, wind energy, and geothermal energy, but these sources lack an adequate supply. Biomass materials, such as mill residues, agricultural crops and wastes, and industrial wastes, have long been used as renewable fuels. Biosolids, on the other hand, have not previously been considered as a renewable energy source due to the large volume of cell-bound water contained therein. As discussed above, the large volume of cell-bound water in biosolids significantly impacts both the cost of incinerating biosolids and the cost of transporting biosolids.

Accordingly, new methods for processing and disposing of organic material are needed.

### SUMMARY

The present invention is directed to processing organic material. In an aspect of the invention, is provided apparatuses and processes for converting organic material into renewable fuel products.

According to an exemplary embodiment of the present invention, a method is provided including the steps of: (a) providing a feedstock that contains organic material; (b) providing a feedstock that contains organic material that that has been processed into a slurry by heating and pressurizing to a pressure that substantially inhibits water in the slurry from vaporizing; (c) at least partially evenly mixing the feedstock of (a) and the slurry of (b); and (d) heating the mixture of (c).

In certain embodiments, the feedstock of (b) is pressurized prior to heating.

In certain embodiments, the method further includes introducing the slurry of (b) to the feedstock of (a) prior to step (c).

In certain embodiments, step (c) occurs in a mechanically-driven mixing device.

In certain embodiments, step (c) occurs in a static mixing device.

In certain embodiments, the feedstock of (a) has been pressurized.

In certain embodiments, step (c) further includes reducing the viscosity of the mixture of (c) to less than 10,000 cp.

In certain embodiments, step (c) further includes raising the temperature of the mixture of (c) to at least 100° F. (38° C.).

In certain embodiments, step (d) further includes raising the temperature of the mixture of (c) to at least 350° F. (177° C.).\

In certain embodiments, step (d) further includes raising the temperature of the mixture of (c) to a temperature below 600° F. (316° C.).

In certain embodiments, subsequent step (d), at least a portion of the mixture of (c) includes the feedstock of (b).

In certain embodiments, step (d) occurs in the presence of at least one of an alkali and a reducing gas to facilitate removal of a constituent.

In certain embodiments, the organic material includes sewage.

In certain embodiments, the organic material includes sewage sludge.

In certain embodiments, the organic material includes biosolids.

In certain embodiments, the feedstock of (a) has not been processed into a slurry by heating.

In certain embodiments, at least one of the feedstock of (a) and the feedstock of (b) contains a chemical agent that facilitates removal of phosphorous.

In certain embodiments, the method further includes mechanically dewatering the mixture of (c).

In certain embodiments, the method further includes thermally drying the mixture of (c).

In certain embodiments, the method further includes solar drying the mixture of (c).

According to an exemplary embodiment of the present invention, a method is provided including the steps of: (a) providing a feedstock containing organic material and ammonia; (b) pressurizing and heating the feedstock to provide a processed slurry; (c) flashing the processed slurry to reduce the pressure and temperature of the processed slurry and to vaporize at least a portion of the ammonia; and (d) recovering the portion of the ammonia vaporized during said step (c).

In certain embodiments, the flashing step (c) occurs after the pressurizing step (b) and prior to the recovering step (d).

In certain embodiments, the flashing of step (c) occurs in a flash tank.

In certain embodiments, step (c) further includes vaporizing water and carbon dioxide.

In certain embodiments, step (d) further includes distilling the vaporized components to recover a liquid stream of water and ammonia.

In certain embodiments, the portion of the ammonia vaporized in step (c) includes between 10% and 70% by weight of the ammonia in the feedstock.

In certain embodiments, step (c) further includes reducing the pressure of the processed slurry from an initial pressure above a water saturation pressure at a temperature of the processed slurry.

In certain embodiments, step (c) further includes reducing the pressure of the processed slurry from an initial pressure below 600 psig.

In certain embodiments, step (c) further includes reducing the pressure of the processed slurry to a pressure less than 25 psig.

In certain embodiments, step (c) further includes reducing the pressure of the processed slurry to a pressure less than 10 psig.

In certain embodiments, step (c) further includes reducing the pressure of the processed slurry to atmospheric pressure.

In certain embodiments, step (c) further includes reducing the pressure of the processed slurry to a pressure above the saturation pressure of water at the reduced temperature.

In certain embodiments, step (c) further includes reducing the temperature of the processed slurry from an initial temperature of at least 300° F. (149° C.).

In certain embodiments, step (c) further includes reducing the temperature of the processed slurry to a temperature less than 250° F. (121° C.).

In certain embodiments, step (b) occurs in the presence of at least one of an alkali and a reducing gas to facilitate removal of a constituent.

In certain embodiments, the organic material includes sewage.

In certain embodiments, the organic material includes sewage sludge.

In certain embodiments, the organic material includes biosolids.

In certain embodiments, the feedstock contains a chemical agent that facilitates removal of phosphorous.

In certain embodiments, the method further includes mechanically dewatering the processed slurry.

In certain embodiments, the method further includes thermally drying the processed slurry.

In certain embodiments, the method further includes solar drying the processed slurry.

According to an exemplary embodiment of the present invention, a method is provided including the steps of: (a) providing a feedstock containing organic material; (b) pressurizing and heating the feedstock to provide a processed slurry; (c) dewatering the processed slurry; and (d) adding a halide to provide a fuel product.

In certain embodiments, the adding step (d) occurs after the dewatering step (c).

In certain embodiments, the halide of step (d) includes at least one of fluoride, chloride, bromide, and iodide.

In certain embodiments, the halide of step (d) includes at least one of NaCl, HCl, HBr, and CaBr₂.

In certain embodiments, the adding step (d) further includes treating the processed slurry with an aqueous halide solution.

In certain embodiments, the method further includes drying the dewatered slurry.

In certain embodiments, step (b) occurs in the presence of at least one of an alkali and a reducing gas to facilitate removal of a constituent.

In certain embodiments, the method further includes combusting the solid fuel product to promote mercury oxidation.

In certain embodiments, the organic material includes sewage.

In certain embodiments, the organic material includes sewage sludge.

In certain embodiments, the organic material includes biosolids.

In certain embodiments, the feedstock contains a chemical agent that facilitates removal of phosphorous.

In certain embodiments, the method further includes thermally drying the processed slurry.

In certain embodiments, the method further includes solar drying the processed slurry.

According to an exemplary embodiment of the present invention, a method is provided including the steps of: (a) providing a feedstock that contains organic material; (b) processing the feedstock into a slurry by heating and pressurizing the slurry to a pressure that substantially inhibits water in the slurry from vaporizing; (c) dewatering the processed slurry to provide a liquid centrate; and (d) digesting to produce methane at least a portion of the processed slurry after the processing of (b) and prior to the dewatering of (c).

In certain embodiments, the method further includes digesting to produce methane the liquid centrate to provide a processed centrate.

In certain embodiments, the method further includes combining at least a portion of the processed centrate with the processed slurry.

In certain embodiments, the digesting of (d) occurs in the presence of a digester feed to enhance production of methane.

In certain embodiments, the digester feed includes at least one of a fat, an oil, or a grease.

In certain embodiments, the organic material includes sewage.

In certain embodiments, the organic material includes sewage sludge.

In certain embodiments, the organic material includes biosolids.

In certain embodiments, the feedstock contains a chemical agent that facilitates removal of phosphorous.

In certain embodiments, the method further includes thermally drying the processed slurry.

In certain embodiments, the method further includes solar drying the processed slurry.

### BRIEF DESCRIPTION OF THE DRAWINGS

The above-mentioned and other features and advantages of this invention, and the manner of attaining them, will become more apparent and the invention itself will be better understood by reference to the following description of embodiments of the invention taken in conjunction with the accompanying drawings, wherein:

FIG. 1 is a schematic diagram illustrating a process for converting organic material into a renewable fuel product;

FIG. 2 is a schematic diagram similar to FIG. 1 illustrating an alternative process for converting organic material into a renewable fuel product; and

FIG. 3 is a schematic diagram illustrating a process for treating vapors exhausted from the process.

Corresponding reference characters indicate corresponding parts throughout the several views. The exemplifications set out herein illustrate exemplary embodiments of the invention and such exemplifications are not to be construed as limiting the scope of the invention in any manner.

### DETAILED DESCRIPTION

The present invention relates to the processing of organic material and, in an embodiment, relates to processes for converting organic material into renewable fuel products.

In an aspect of the invention, feedstock containing organic material is processed at an elevated pressure and temperature, causing cell structures to undergo lysing, decarboxylation, and carbonization.

In a further aspect of the invention, a portion of the processed slurry may be recirculated through the process and mixed with cool, heated or pressurized feedstock prior to reaching a mechanical mixing device to preheat the feedstock and to reduce the viscosity of the feedstock.

In a further aspect of the invention, the pressure and temperature of the processed slurry are then reduced. This pressure and temperature reduction may be performed simultaneously to flash volatile materials, such as ammonia, out of the slurry and into a vapor phase, thereby reducing the amount of those materials present in the final product and allowing the materials to be recovered from the process.

In a further aspect of the invention, after letting down the pressure and temperature, the processed slurry may be treated with a halide to reduce mercury emissions in the final product. The processed slurry is then subjected to mechanical and thermal dewatering processes, resulting in a renewable fuel product in dried particulate or pelletized form. Unlike the incoming feedstock, the final product is a viable energy source having a positive heating value.

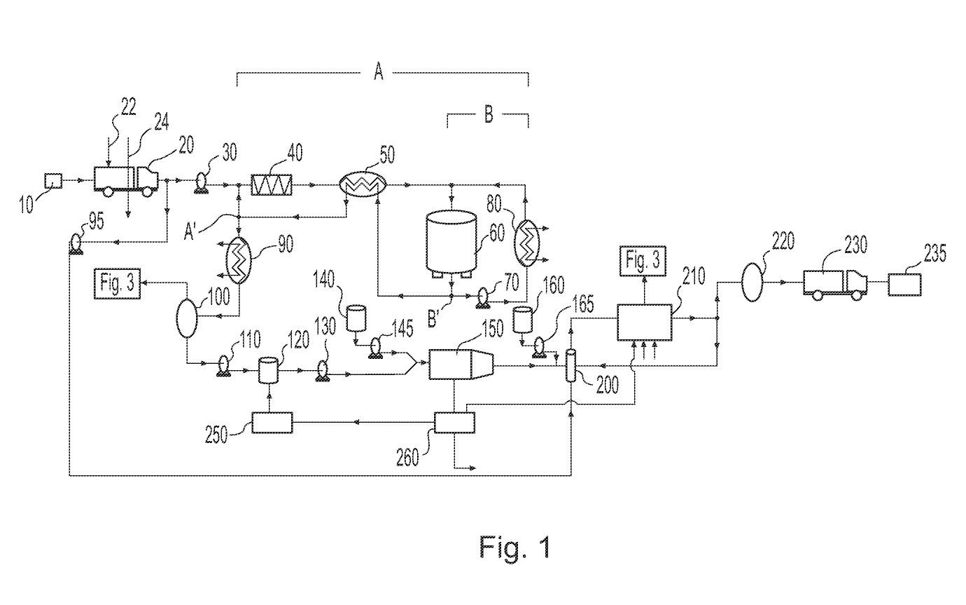

Referring to FIG. 1, a process is illustrated for converting organic material into viable renewable fuel products. A feedstock containing organic material is received from a client, such as wastewater treatment plant (WWTP) 10. The feedstock received from WWTP 10 may include sewage in the form of a sludge. For example, the feedstock received from WWTP 10 may include untreated sewage sludge or processed sewage sludge, such as sludge containing Class A or Class B biosolids. The term “biosolids” as used throughout this disclosure has its ordinary meaning in the art. For example, biosolids include dead organic cells, bacterial cell masses, inorganic compounds (i.e. grits), cell-bound water, soil-like residue of materials removed from sewage during the wastewater treatment process and other solids.

Prior to leaving WWTP 10, the feedstock may be macerated to reduce the size of solid particles contained in the feedstock. The feedstock may also be subjected to mechanical dewatering processes, such as centrifuges, belt presses, and rotary presses. The feedstock may also be subjected to polymer treatment processes, chemical treatment processes, such as being mixed with a chelating agent, and biological treatment processes, including anaerobic and aerobic digestion processes. In an embodiment, even after undergoing such pretreatments, the feedstock received from WWTP 10 may include as little as approximately 3%, 5%, 10%, or 15% solids by volume or as much as approximately 20%, 25%, 30%, or 35% solids by volume in the form of dead organic cells, bacterial cell masses, and other solids, with cell-bound water making up the remaining volume of the feedstock. The feedstock may also include dissolved substances such as ammonia nitrogen (NH₃—N).

The present invention is also applicable to the processing of other organic materials, especially those containing cell-bound water. For example, the feedstock may include paper mill sludge, food waste, plant matter (such as rice hulls and hay straw), discarded cellulosic packaging material, bagasse, green waste (such as leaves, clippings, and grass), algae, wood and wood waste, clinker or other residue from combustion of wood, palm oil residue and short rotation crops. The feedstock may also include animal carcasses. The feedstock may also include agricultural waste such as sewage material obtained from the live stock industry, for example, hog manure, chicken litter, or cow manure. The feedstock may also include crops grown specifically for use in the process such as switch grass or other plants. The feedstock may also include municipal solid waste, fats oils, and greases (FOG), medical waste, paper waste, refuse derived fuels, Kraft Mill black liquor or hydrophilic non-renewable fuels, such as low-rank coals. In an exemplary embodiment, the feedstock may include a blend of biosolids and other organic materials, including biomass, to enhance the heating value of the final product and/or increase the scale of production.

Referring still to FIG. 1, the feedstock is delivered from WWTP 10, such as via truck 20. The feedstock may also be delivered from WWTP 10 via pipes, other conduits, or another suitable mode of transport, such as a train or barge.

After delivering the feedstock, truck 20 may be subjected to both internal rinse 22 and external wash 24 of water. The source of water for internal rinse 22 and external wash 24 is, for example, service water from the process. Internal rinse 22 may include approximately 25 gallons of service water per truck 20. The effluent from internal rinse 22 remains within the process so that organic material taken into internal rinse 22 becomes part of the process. External wash 24 may include approximately 100 gallons of service water per truck 20. Unlike internal rinse 22, the effluent from external wash 24 exits the process.

The incoming feedstock, and optionally the effluent of internal rinse 22, may next be generated into a slurry, referred to herein as the feedstock slurry. For example, additional water may be added to the feedstock to reduce its viscosity to a more easily pumpable level, such as a viscosity as low as approximately 10,000 centipoise (cp), 20,000 cp, or 30,000 cp or as high as approximately 90,000 cp, 100,000 cp, or 110,000 cp. Also, the incoming feedstock may be blended for uniformity. Further, the incoming feedstock may undergo sieving, maceration, screening, mechanical dewatering, slurrying, or a combination thereof. The incoming feedstock may also be mixed with chelating agents or other chemicals to enhance subsequent removal of constituents such as phosphorous. It is also within the scope of the present invention that the feedstock may be received from WWTP 10 without requiring further processing. The incoming feedstock may be held in a holding tank before continuing to high pressure pump 30.

Optionally, before reaching high pressure pump 30, which will be described in more detail below, a portion of the feedstock slurry may be bypassed to mixer 200 of dryer 210 via bypass pump 95. This portion of the feedstock slurry may be bypassed to dryer 210 to take advantage of excess capacity within dryer 210. Therefore, the portion of feedstock slurry that is bypassed may depend on the size of dryer 210.

The remaining feedstock slurry not bypassed to dryer 210 continues to high pressure pump 30. The feedstock slurry is pumped to a pressure above the saturation pressure of water to maintain a liquid phase during subsequent heating operations by substantially inhibiting water in the slurry from vaporizing. The feedstock slurry may be pressurized at pump 30 to a pressure as low as the saturation pressure of water at the subsequent elevated slurry temperature, 200 psig, 300 psig, 400 psig, 500 psig, 600 psig, 700 psig, or 800 psig or as high as approximately 900 psig, 1000 psig, 1100 psig, or 1200 psig, for example. The pressure supplied by pump 30 may vary depending on the viscosity of the feedstock slurry. As the viscosity of the feedstock slurry increases, the pressure supplied by pump 30 may be increased to account for downstream pressure loss. Care must be exercised to provide pump 30 with an adequate net pump suction head (NPSH), either hydraulically or by mechanical assistance, considering that the feedstock slurry may be very viscous and may carry dissolved gases. In an embodiment, the pressurized feedstock slurry may subsequently travel along a horizontal or downward-sloping plane in order to, with assistance from the Earth's gravitational force, reduce the demand on pump 30 and/or the likelihood of gritty or sticky solid portions of the slurry collecting in the process, such as within pipes of the process.

The pressurized feedstock slurry is then heated. In an exemplary embodiment of the present invention, illustrated in FIG. 1, the pressurized feedstock slurry is heated by a combination of heat exchange devices and slurry recirculation loops. The illustrated embodiment includes external slurry recirculation loop A that recirculates processed slurry through reactor 60 via first heat exchanger 50. Within external slurry recirculation loop A, the illustrated embodiment also includes internal slurry recirculation loop B that recirculates processed slurry through reactor 60 via second heat exchanger 80. The use of external slurry recirculation loop A, internal slurry recirculation loop B, first heat exchanger 50, and second heat exchanger 80 to heat the pressurized feedstock slurry is described in more detail below.

From pump 30, the pressurized feedstock slurry continues to mixer 40. However, before entering mixer 40, the feedstock slurry from pump 30 may be mixed with hot, processed slurry from reactor 60 traveling in external slurry recirculation loop A. This process of mixing feedstock slurry with processed slurry will be discussed in more detail below. In an embodiment of the present invention, mixer 40 may include an in-line, static mixing device. By providing an in-line, static mixing device, the pressure loss across mixer 40 may be minimized, so as to maintain the pressure supplied by pump 30. A static mixer 40 may include, for example, static agitators, obstacles, spirals, or protrusions to promote even mixing of the feedstock slurry and the processed slurry by creating turbulence in the slurry, In another embodiment of the present invention, mixer 40 may include an in-line, mechanically driven mixing device. A mechanically driven mixer 40 may include, for example, a moving agitator, such as a screw, a blade set, or another suitable agitator to promote even mixing of the feedstock slurry and the processed slurry by creating turbulence in the slurry. Mixer 40 is discussed further below.

Referring still to FIG. 1, to increase the temperature of the pressurized feedstock slurry, the feedstock slurry is directed to first heat exchanger 50. Before reaching first heat exchanger 50, the feedstock slurry from mixer 40 may have already undergone heating due to mixing with hot, processed slurry from reactor 60, as will be discussed in more detail below. In that case, the slurry entering first heat exchanger 50 may already have a temperature as high as approximately 235° F. (113° C.) or more. First heat exchanger 50 is optional and may be provided to further increase the temperature of the pressurized feedstock slurry by exchange with a heat transfer fluid. In an exemplary embodiment of the present invention, the heat transfer fluid of first heat exchanger 50 is hot, processed slurry exiting reactor 60. After exiting first heat exchanger 50, the feedstock slurry may increase in temperature by approximately 35° F. (19° C.) or more. For example, the feedstock slurry may increase in temperature from approximately 235° F. (113° C.) to approximately 270° F. (132° C.) or more.

At this elevated temperature, the slurry exiting first heat exchanger 50 may begin to undergo cell lysing, in which the walls, membranes and/or other aspects of cell structures in the feedstock rupture. For example, the cell walls, cell lipid-bilayer membranes and/or internal cellular membranes of dead organic matter in biosolids may rupture and the cells may break down into particles of smaller size, thereby releasing cell-bound water from the biosolids. This lysing process typically begins at a temperature of approximately 230° F. (110° C.). Due to heating, cell lysing, and the release of cell-bound water, the viscosity of the slurry exiting first heat exchanger 50 may be reduced. For example, the slurry exiting first heat exchanger 50 may have a viscosity as low as approximately 100 cp, 500 cp, 1000 cp, or 2,000 cp or as high as approximately 4,000 cp, 6,000 cp, 8,000 cp, or 10,000 cp. The rupturing of cells may also cause impurities, such as sodium, potassium, chlorine, sulfur, nitrogen, toxic metals, and other impurities, to separate from the cell structures as anions and cations and dissolve in the liquid phase. The separation and dissolution of impurities as anions and cations makes such molecules accessible to subsequent removal or disposal. Similarly, undesirable constituents may take gaseous forms which may be more conveniently removed in subsequent processes. The rupturing of cells may also allow for more efficient generation of methane in subsequent digestion processes.

To still further increase the temperature of the pressurized slurry before entering reactor 60, the slurry may be mixed with hot slurry from second heat exchanger 80 traveling in internal slurry recirculation loop B. The mixing of hot slurry from second heat exchanger 80 with slurry from first heat exchanger 50 will be discussed in more detail below. It is also within the scope of the present invention that the slurry exiting first heat exchanger 50 may travel through second heat exchanger 80 before entering reactor 60.

Referring still to FIG. 1, reactor 60 is provided so that the organic material, such as biosolids, contained in the incoming feedstock will continue to undergo lysing, as well as decarboxylation and optionally carbonization. Inside reactor 60, the hot, pressurized slurry is subjected to a dwell time such that the cell structures continue to rupture and break down into particles of smaller size, thereby releasing more cell-bound water. The rupturing of cells may also continue to cause impurities, such as sodium, potassium, chlorine, sulfur, nitrogen, toxic metals, protoplasm, proteins, other cell contents, and other impurities, to separate from the cell structures as anions and cations and dissolve in the liquid phase. The separation and dissolution of impurities as anions and cations makes such molecules accessible to subsequent removal or disposal. Similarly, undesirable constituents may take gaseous forms which may be more conveniently removed in subsequent processes. The continued rupturing of cells as well as decarboxylation may also allow for more efficient generation of methane in subsequent digestion processes. As mentioned above, lysing typically begins at a temperature of approximately 230° F. (110° C.), increases at a substantially exponential rate with increasing temperature, and is typically complete upon reaching a temperature of approximately 300-320° F. (149-160° C.).

Continued presence in reactor 60 and continued heating of the slurry causes oxygen to split off from the ruptured cells in the form of carbon dioxide, referred to as decarboxylation. At even higher temperatures, the ruptured cells carbonize, which converts the cell constituents to char. Decarboxylation and carbonization occur at higher temperatures than lysing, such as between approximately 350-600° F. (177-316° C.).

Reactor 60 may be designed to achieve varying degrees of lysing, decarboxylation, and carbonization depending on the desired output. As the temperature, pressure, and/or dwell time of the slurry in reactor 60 is increased, the slurry may undergo more complete lysing, decarboxylation, and carbonization. For example, if small particles are desired in the output, the temperature, pressure, and/or dwell time of the slurry in reactor 60 may be increased to promote complete lysing of the cells. The temperature of the slurry in reactor 60 may also be designed to reduce or destroy human pathogens. This process may be optionally carried out in the presence of an alkali, reducing gas, or other compounds to facilitate the removal of undesirable constituents. For example, the process may be carried out in the presence of carbon monoxide to facilitate the removal of precipitated ammonia.

Reactor 60 should be capable of handling the incoming flow of slurry over the allotted dwell time. The dwell time in reactor 60 may be 1 minute, 2 minutes, 3 minutes, 4 minutes, or more. As mentioned above, varying degrees of lysing, decarboxylation, and carbonization may be achieved by altering the dwell time inside reactor 60, such as by changing the size of reactor 60. The incoming flow of slurry may proceed downward through reactor 60 to enhance the removal of sand, grit, and other materials from the slurry, which will collect in the bottom of reactor 60. Reactor 60 may be a continuous stirred-tank reactor (CSTR). While a continuous reaction within reactor 60 is disclosed herein, the present invention also contemplates a batch reaction, as described in more detail below.

Referring still to FIG. 1, the slurry may enter reactor 60 at a temperature as low as approximately 350° F. (177° C.), 375° F. (191° C.), 400° F. (204° C.), or 425° F. (218° C.) or as high as approximately 525° F. (274° C.), 550° F. (288° C.), 575° F. (302° C.), or 600° F. (316° C.), for example. As mentioned above, varying degrees of lysing, decarboxylation, and carbonization may be achieved by altering the temperature of the slurry in reactor 60, such as by changing the temperature of the incoming slurry. Reactor 60 may itself be heated to further heat the slurry or to maintain the slurry at the incoming temperature. In this embodiment, a jacket of reactor 60 may receive heat transfer fluid, such as Dow Therminol 59, at an inlet temperature of approximately 500° F. (260° C.) to maintain the slurry at a temperature of approximately 405° F. (207° C.). Similarly, reactor 60 could be heated by direct steam injection, heating coils, or a combination thereof. As known to those of ordinary skill in the art, the methods for heating continuous reactors may be similar to those for batch reactors. It is also within the scope of the present invention that the slurry reactions will generate heat in reactor 60, thereby reducing the amount of heat added to reactor 60.

The slurry may enter reactor 60 at a pressure that substantially inhibits water in the slurry from vaporizing. In an embodiment, the slurry may enter reactor 60 at a pressure as low as the saturation pressure of water at the current slurry temperature, 150 psig, 200 psig, 250 psig, 300 psig, 350 psig, or 400 psig or as high as approximately 500 psig, 550 psig, 600 psig, 650 psig, 700 psig, 750 psig, or 800 psig, for example. As mentioned above, varying degrees of lysing, decarboxylation, and carbonization may be achieved by altering the pressure of the slurry in reactor 60, such as by changing the pressure supplied by pump 30. The pressure in reactor 60 depends on the pressure supplied by pump 30. Additionally, the pressure in reactor 60 depends on any pressure loss that may occur between pump 30 and reactor 60 due to, for example, the distance traveled between pump 30 and reactor 60 and the viscosity of the slurry.

As mentioned above, the present invention also contemplates a batch reaction within reactor 60. Compared to the continuous reaction described above, a batch reaction may reduce the degree of pumping required to transport the viscous, unprocessed slurry. In this embodiment, the incoming unprocessed slurry may be directed to reactor 60, a chamber capable of being pressurized and heated. Like the continuous process described above, reactor 60 may be designed to achieve varying degrees of lysing, decarboxylation, and carbonization depending on the desired output. As the temperature, pressure, and/or dwell time of the slurry in reactor 60 is increased, the slurry may undergo more complete lysing, decarboxylation, and carbonization.

The slurry leaving reactor 60, referred to herein as processed slurry, contains a mixture of liquid and solid materials. The liquid phase includes primarily water released from the cell structures during lysing, degradation products of the cells and their contents, as well as dissolved carbon dioxide, dissolved NH₃—N, and other volatile materials such as mercury and sulfur compounds. Volatile materials may be forced to remain in the liquid phase due to the high pressure supplied by pump 30. However, some gases may form in the process. To prevent the evolved gases from accumulating in the piping and equipment, the evolved gases may be continuously removed from vents located throughout the process. For example, vents may be located in reactor 60, at high points in the process, and in confined areas, such as centrifugal pump casings, having localized pressure drops that allow dissolved gases to evolve from the slurry. The solid phase includes primarily char and inorganic compounds (i.e. grits). The processed slurry leaving reactor 60 may contain as little as approximately 10%, 20%, or 30% solid materials by weight or as much as approximately 40% or 50% solid materials by weight. The solid content of the processed slurry may be somewhat less than the solid content of the unprocessed slurry due to the release of bound oxygen, carbon, and other materials into the liquid and gaseous phases, as well as chemical reactions among the constituents.

The solid particles in the processed slurry may have various sizes. In an exemplary embodiment of the present invention, approximately 95% or more of the solid particles in the processed slurry may be less than 200 micrometers in size or less than 150 micrometers in size. Further, approximately 50% or more of the solid particles in the processed slurry may be less than 50 micrometers in size, 20 micrometers in size, or 10 micrometers in size. Table 1 below sets forth an exemplary size distribution of solid particles in the processed slurry.

TABLE 1

| Particle Size | Particles under |
|---|---|
| (micrometers) | Particle Size |
| 149 | 96% |
| 74 | 83% |
| 53 | 76% |
| 44 | 71% |
| 37 | 68% |
| 25 | 60% |
| 20 | 55% |
| 15 | 49% |
| 10 | 40% |
| 5 | 22% |
| 3 | 10% |

The processed slurry leaving reactor 60 undergoes a significant reduction in viscosity. For example, the processed slurry may have a viscosity as low as approximately 30 cp, 100 cp, or 170 cp or as high as approximately 1200 cp, 1650 cp, or 2600 cp. For example, Table 2 below sets forth the experimental viscosity of the processed slurry at 177° F. (81° C.) and at variable shear rates.

TABLE 2

| Shear Rate | Viscosity |
|---|---|
| (sec⁻¹) | (cp) |
| 1.53 | 1073 |
| 3.06 | 644 |
| 5.11 | 442 |
| 10.21 | 265 |
| 51.07 | 81 |
| 102.12 | 49 |
| 153.21 | 36 |
| 170.23 | 33 |
| 306.41 | 22 |
| 340.46 | 20 |
| 510.69 | 15 |
| 1021.38 | 9 |

Referring still to FIG. 1, at point B′, a portion of the processed slurry may be recirculated through reactor 60 via internal slurry recirculation loop B mentioned above. In an exemplary embodiment of the present invention, as little as 40%, 45%, or 50% of the processed slurry by volume or as much as 55%, 60%, and 65% of the processed slurry by volume may be directed to recirculation pump 70, which then directs the processed slurry through second heat exchanger 80 and through reactor 60. In second heat exchanger 80, the temperature of the processed slurry may be raised by approximately 65° F. (36° C.) or more. For example, the temperature of the processed slurry may be raised from approximately 405° F. (207° C.) to approximately 470° F. (243° C.). Second heat exchanger 80 may heat the processed slurry by exchange with a heat transfer fluid. In the present embodiment, second heat exchanger 80 may receive heat transfer fluid, such as Dow Therminol 59, at an inlet temperature exceeding 575° F. (302° C.). The heat transfer fluid may be the same as that sent to reactor 60 to maintain the temperature of the slurry during the reaction. The processed slurry from second heat exchanger 80 is combined with slurry from first heat exchanger 50, and then the combined stream enters reactor 60. In an exemplary embodiment of the present invention, the inlet to reactor 60 may include a mixture of approximately 40% slurry by volume from first heat exchanger 50 at a temperature of approximately 270° F. (132° C.) and approximately 60% slurry by volume from second heat exchanger 80 at a temperature of approximately 470° F. (243° C.), resulting in a blended temperature of approximately 405° F. (207° C.). This blended temperature may be increased or decreased to optimize the plant by adjusting the recycle ratios and/or the heat applied to the heaters.

At point B′, a remaining portion of the processed slurry not recirculated through reactor 60 via internal slurry recirculation loop B continues through the process. In an exemplary embodiment of the present invention, the remaining portion of the processed slurry may continue to first heat exchanger 50 to undergo cooling. Cooling the processed slurry in first heat exchanger 50 serves the additional purpose of heating the feedstock slurry from mixer 40. The processed slurry may enter first heat exchanger 50 at the outlet temperature of reactor 60, such as approximately 405° F. (207° C.). After contacting the cool, feedstock slurry from mixer 40, the processed slurry may be cooled by approximately 35° F. (19° C.) or more.

Referring still to FIG. 1, at point A′, a portion of the processed slurry may be recirculated through reactor 60 via external slurry recirculation loop A mentioned above. In an exemplary embodiment of the present invention, as little as 30%, 40%, or 50% of the processed slurry by volume or as much as 60% or 70% of the processed slurry by volume from first heat exchanger 50 may be recirculated and introduced to feedstock slurry received from WWTP 10 downstream of pump 30 and upstream of mixer 40. As discussed below, the portion of the processed slurry that is recycled at point A′ determines the mixing ratio of processed slurry to feedstock slurry, which may vary between about 1 to 1 and about 5 to 1. As mentioned above, mixer 40 may include, for example, an in-line, static mixing device or an in-line, mechanically-driven mixing device. Mixer 40 promotes even mixing of the feedstock slurry and the processed slurry via turbulence. For example, obstacles within a static mixing device may divert the flow of the slurry streams. Without mixer 40, the thin, less viscous, processed slurry may not blend with the thick, more viscous, feedstock slurry to form a combined stream. For example, without mixer 40, the feedstock slurry may collect on pipe walls and the processed slurry may travel inside the collected feedstock slurry. The combination of the processed slurry with the feedstock slurry in mixer 40 is important for at least three reasons.

First, the combination of the processed slurry with the feedstock slurry recovers heat from the processed slurry and preheats the feedstock slurry. This mixing reduces the heat energy requirements for the process. The temperature of the combined slurry stream may be controlled by varying the mixing ratio of processed slurry to feedstock slurry. For example, the mixing ratio may vary between about 1 to 1 and about 5 to 1 such that the combined slurry stream may have a temperature as low as approximately 100° F. (38° C.), 150° F. (66° C.), or 200° F. (93° C.) or as high as approximately 250° F. (121° C.), 300° F. (149° C.), or 350° F. (177° C.). Depending on the desired temperature of incoming slurry to reactor 60, the recirculation of processed slurry could reduce the heat to be supplied by first heat exchanger 50 or could eliminate the need for first heat exchanger 50 altogether. The ability to meet the desired temperature of the combined slurry stream is dependent on achieving adequate mixing of the processed slurry and the feedstock slurry in mixer 40.

Second, the combination of the processed slurry with the feedstock slurry substantially reduces the viscosity of the feedstock slurry stream and thus the required pumping power and the associated operating costs. The reduced viscosity may accommodate use of centrifugal pumps rated at pressures up to 500 psi. Also, the reduced viscosity allows pump 30 to maintain a high pressure condition throughout the system and reduces the potential for fouling within the system, including mixer 40. The viscosity of the combined slurry stream may be controlled by varying the mixing ratio of processed slurry to feedstock slurry. For example, the mixing ratio may vary between about 1 to 1 and about 5 to 1 such that the combined slurry stream may have a viscosity as low as approximately 100 cp, 500 cp, 1000 cp, or 2,000 cp or as high as approximately 4,000 cp, 6,000 cp, 8,000 cp, or 10,000 cp. As mentioned above, mixer 40 promotes this reduction in viscosity by blending the thin, less viscous, processed slurry with the thick, more viscous, feedstock slurry to form a combined slurry stream rather than separate slurry streams having varying viscosities. Thus, the ability to meet the desired viscosity of the combined slurry stream is dependent on achieving adequate mixing of the processed slurry and the feedstock slurry in mixer 40.

Third, the combination of the processed slurry with the feedstock slurry preheats and reduces the viscosity of the feedstock slurry without substantially decreasing the solids content of the combined stream. The addition of hot water, for example, to the feedstock slurry may preheat and reduce the viscosity of the feedstock slurry. However, the addition of hot water would also dilute the solids content of the combined stream.

At point A′, a remaining portion of the processed slurry not recirculated through reactor 60 via external slurry recirculation loop A exits the slurry recirculation loops. The processed slurry then continues through the process.

The embodiment of heating the pressurized slurry described above and illustrated in FIG. 1 may be optionally altered to achieve several objectives. First, the embodiment may be altered to achieve a desired temperature in reactor 60. For example, more processed slurry from reactor 60 may be recirculated through internal slurry recirculation loop B to second heat exchanger 80 to increase the temperature of the incoming slurry to reactor 60. In other words, the incoming slurry to reactor 60 may contain more heated slurry from second heat exchanger 80 and less partially heated slurry from first heat exchanger 50. Also, the arrangement may be altered to optimize the amount of energy necessary to heat the slurry. For example, more processed slurry from reactor 60 may be recirculated through external slurry recirculation loop A to first heat exchanger 50 to reduce the energy that must be supplied to first heat exchanger 50.

After exiting the slurry recirculation loops described above and before undergoing mechanical dewatering, the processed slurry is depressurized and cooled. The processed slurry may be depressurized and cooled by various methods.

According to a first embodiment of the present invention, illustrated in FIG. 1, the processed slurry may travel from first heat exchanger 50 to third heat exchanger 90, in which its temperature may be lowered by exchange with plant cooling water, for example. Third heat exchanger 90 may be capable of decreasing the temperature of the reacted slurry by approximately 150° F. (83° C.) or more. After being cooled in third heat exchanger 90, the processed slurry then enters pressure letdown tank 100. In pressure letdown tank 100, the pressure of the processed slurry drops to a pressure as low as atmospheric pressure, 5 psig, or 10 psig or as high as approximately 15 psig, 20 psig, or 25 psig, for example. Because foaming may occur in pressure letdown tank 100, it may be advantageous to control foaming by using a spray nozzle from the lower part of pressure letdown tank 100 to spray a side stream into pressure letdown tank 100. The reduction in pressure liberates volatile materials once forced to remain in the liquid phase, such as carbon dioxide, hydrogen sulfide, and other non-condensable gases. Pressure letdown tank 100 may also liberate some small amounts of water vapor, but remaining liquid water not vaporized in pressure letdown tank 100 may be removed during subsequent mechanical dewatering and thermal drying processes. Most or all of the dissolved NH₃—N may remain dissolved within the liquid phase. Pressure letdown tank 100 may also be used to release vent gases that evolved elsewhere in the process. For example, vent piping may connect reactor 60 to pressure letdown tank 100 to release gases that evolved in and were vented from reactor 60, along with the other gases that evolved in pressure letdown tank 100.

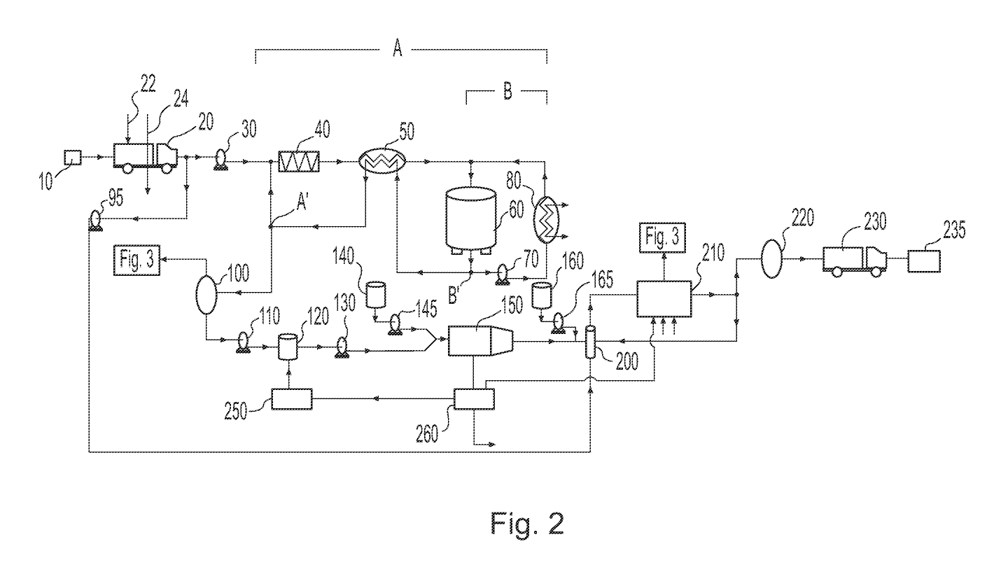

According to another embodiment of the present invention, illustrated in FIG. 2, the hot, processed slurry may travel from first heat exchanger 50 to pressure letdown tank 100, without first entering third heat exchanger 90. It is also within the scope of the present invention that the hot, processed slurry may travel from reactor 60 to pressure letdown tank 100, without first entering first heat exchanger 50 or third heat exchanger 90. In this embodiment, the processed slurry may enter pressure letdown tank 100 at a temperature as low as approximately 300° F. (149° C.), 350° F. (179° C.), or 400° F. (204° C.) or as high as approximately 450° F. (232° C.), 500° F. (260° C.), or 550° F. (288° C.), for example. In pressure letdown tank 100, the pressure of the processed slurry may drop to a pressure as low as atmospheric pressure, 5 psig, or 10 psig or as high as approximately 15 psig, 20 psig, or 25 psig, for example. This reduced pressure may vary depending on the desired composition of the liberated vapor. For example, the pressure may be sufficiently low to vaporize ammonia while being sufficiently high to maintain water in the liquid phase to be removed during subsequent dewatering processes. The pressure reduction causes the slurry and vapor to simultaneously cool to the saturation temperature of the liquid at the reduced pressure. Depending on the pressure of pressure letdown tank 100, the temperature of the processed slurry may drop to a temperature as low as approximately 75° F. (24° C.), 100° F. (38° C.), or 125° F. (52° C.) or as high as approximately 200° F. (93° C.), 225° F. (107° C.), or 250° F. (121° C.), for example. In this embodiment, it may be advantageous to recover the heat lost from the slurry directly from pressure letdown tank 100, rather than being recovered from third heat exchanger 90 as in the previous embodiment. Because foaming may occur in pressure letdown tank 100, it may also be advantageous to control foaming by using a spray nozzle from the lower part of pressure letdown tank 100 to spray a side stream into pressure letdown tank 100.

The simultaneous reduction in pressure and temperature described in the previous paragraph, referred to as a flash separation, liberates volatile materials once forced to remain in the liquid phase, such as carbon dioxide, hydrogen sulfide, mercaptans, and other non-condensable gases. The reduction in pressure and temperature in pressure letdown tank 100 may liberate additional volatile materials such as NH₃—N, mercury, and orthophosphates. Ammonia (NH₃) exists in equilibrium with the water in the slurry. The solubility of ammonia in the water depends on the temperature and pressure of the system. According to Henry's Law (Equation 1), the concentration c of ammonia in the water is proportional to the partial pressure p of ammonia above the water at a particular temperature, where k_{H} is a constant for the particular temperature with the dimensions of pressure divided by concentration.

p=k_{H}c (Equation 1)

The particular ammonia (NH₃) species present in the system and the partition between the ammonia (NH₃) and ammonium (NH₄) species depends on the pH of the system. In an exemplary embodiment, the pH of the system is at or near neutral, such that both ammonia and ammonium species are readily dissolved in the water. By evaporating water in pressure letdown tank 100, the ammonia and ammonium species dissolved therein are also vaporized and re-equilibrate to the new process thermodynamic state. In an exemplary embodiment of the present invention, approximately 10%, 20%, 30%, 40%, 50%, 60%, 70%, or more of the ammonia species by weight in the process may be exhausted from pressure letdown tank 100 by flashing a similar fraction of water from pressure letdown tank 100. Removing these volatile materials, including NH₃—N, from pressure letdown tank 100 is important for at least two reasons. First, removing volatile materials from pressure letdown tank 100 reduces the amount of those materials present in the final product. For example, removing NH₃—N from the final product may make the final product more suitable for subsequent combustion, such as in cement production processes. Also, removing volatile materials from pressure letdown tank 100 may allow those materials to be recovered and sold, as described in the following paragraph.

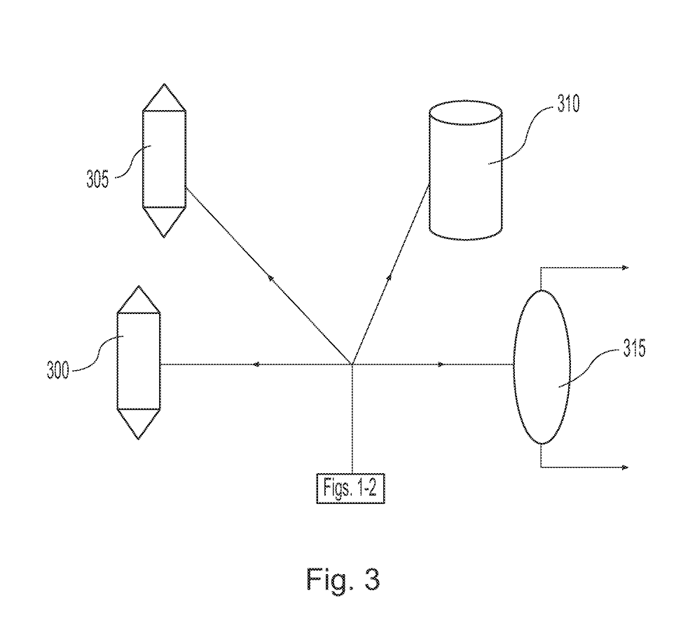

In both of the previously described embodiments, illustrated in FIGS. 1 and 2, the liberated vapor stream separates from the liquid and solid materials and is removed from the top of pressure letdown tank 100. The separated vapor stream removed from the top of pressure letdown tank 100 may comprise a mixture of carbon gas, water vapor, NH₃—N, mercury, orthophosphates, and/or other volatile materials. As shown in FIG. 3, the vapors exiting pressure letdown tank 100 may be captured, purified, and sold, burned to destroy odors, burned for energy recovery, processed to destroy undesirable components, or otherwise processed. For example, the separated vapor stream may be directed to ammonia scrubber 300 and/or sodium hydroxide scrubber 305. Also, the separated vapor stream may be directed to a regenerative thermal oxidizer 310. In an exemplary embodiment of the present invention, in which the separated vapor stream comprises primarily carbon dioxide gas, water vapor, and NH₃—N, the stream may be directed from the top of pressure letdown tank 100 to a distillation column 315 to produce a liquid stream of water and dissolved NH₃—N. The liquid stream from distillation column 315 may be sold and used, for example, as a fuel, a chemical reagent, a fertilizer, and/or a disinfectant.

Referring again to FIG. 1, the liquid and solid slurry is removed from the bottom of pressure letdown tank 100. Pump 110 may direct the liquid and solid slurry to centrifuge feed tank 120 where it is stored. Remaining gas that failed to vent from pressure letdown tank 100 may vent from centrifuge feed tank 120. For example, remaining NH₃—N that did not exit the process from pressure letdown tank 100 may evaporate in centrifuge feed tank 120. This gas may be processed by an odor gas process and/or any of the gas treatment processes set forth above with reference to FIG. 3. In or after centrifuge feed tank 120, the slurry may receive a polymer feed from polymer tank 140 via polymer feed pump 145 to promote separation of the solid materials and the liquid materials in downstream dewatering processes.

In one or more of the aspects described herein, prior to being mechanically dewatered, at least a portion of the slurry may be digested. According to an embodiment of the present invention, in addition to receiving a polymer feed from polymer tank 140, the slurry in centrifuge feed tank 120 may also receive a feed from dissolved air flotation/cavitation air flotation (DAF/CAF) thickening system 250 and/or digestion system 260. DAF/CAF thickening system 250 may be provided to collect solid materials exhausted from the process, treat those solid materials, and return those solid materials to the process. These solid materials may be recovered in DAF/CAF thickening system 250 after being exhausted from, for example, ammonia scrubber 300 and/or digestion system 260, which is described in more detail below. According to another embodiment of the present invention, the slurry removed from pressure letdown tank 100 may itself be digested prior to being mechanically dewatered. For example, aerobic or anaerobic digestion may occur in centrifuge feed tank 120 or in another suitable system, such as digestion system 260. In digestion system 260, volatile solids and/or organic compounds such as the organic fraction of the slurry to a methane rich gas, normally referred to as biogas. The methane concentration of the biogas may be as low as 50% percent by volume or as high as 70% percent by volume depending on the type of organic feed. The balance of the biogas includes primarily carbon dioxide. According to an exemplary embodiment of the present invention, the digestion process occurs under anoxic conditions and in a two-step biological process whereby two classes of bacteria are used to convert the organic fraction of the slurry into a methane rich biogas. First, a class of bacteria known as acidfiers or acidfying bacteria may be used to hydrolyze the complex organics into volatile fatty acids, such as acetic acid or propionic acid. Second, a class of bacteria known as methanogens may be used to convert the volatile fatty acids into methane rich biogas. According to an exemplary embodiment of the present invention, a portion of the slurry may be digested to produce biogas and the remaining slurry may continue through the process to produce a solid fuel product. The biogas generated in digestion system 260 may be used as fuel in the process, such as fuel for dryer 210. The biogas may also be refined, such as by amine adsorption, pressure swing adsorption, or water wash, to remove carbon dioxide, water, and hydrogen sulfide, thereby producing a purified methane stream known as natural gas The natural gas may be sold and used in industrial boilers and furnaces, for example. Also, prior to being mechanically dewatered, the slurry may undergo solar drying. Solar drying may reduce and/or eliminate the need for a subsequent thermal drying process.

The liquid and solid slurry, optionally including a polymer to promote flocculation, may be directed from centrifuge feed tank 120 via, for example, centrifuge feed pump 130 to at least one mechanical dewatering device. One or more commercially-available devices may be employed to separate the liquid materials from the solid materials in the slurry stream, such as centrifuge 150. Other suitable separation devices include, but are not limited to, thickeners, pressure and vacuum rotary filters, horizontal filters, belt and rotary presses, and similar devices, as well as any combination thereof. The slurry entering centrifuge 150 includes primarily liquid materials, with solid materials making up as little as approximately 5%, 10%, 15%, or 20% of the slurry weight or as much as approximately 25%, 30%, 35%, or 40% of the slurry weight, for example. Remaining NH₃—N that entered the process with the feedstock and that did not escape from pressure letdown tank 100 or vent from centrifuge feed tank 120 enters centrifuge 150 dissolved in the liquid stream.

Upon entering centrifuge 150, the slurry is subjected to high speed rotation to separate the liquid materials from the solid materials. Most of the liquid materials will exit centrifuge 150 in the liquid centrate stream, and most of the solid materials will exit centrifuge 150 in the cake. This stream may contain trace amounts of solid materials. For example, the liquid centrate stream may contain approximately 0.10% solids by weight. Because NH₃—N enters centrifuge 150 in the liquid stream, a majority of the NH₃—N will exit centrifuge 150 in the liquid centrate stream.

The liquid centrate stream exiting centrifuge 150 may undergo subsequent processing to recover solid materials, to produce biogas, and/or to otherwise treat the liquid centrate. According to an exemplary embodiment of the present invention, the liquid centrate stream may be directed to digestion system 260, which may be an aerobic or anaerobic digestion system. Aerobic digestion is a bacterial process occurring in the presence of oxygen, while anaerobic digestion is a process in which microorganisms break down biodegradable material in the absence or the substantial absence (facultative anaerobes) of oxygen. Such centrate digestion may take place in a process where the processed slurry has been digested in a previous step prior to dewatering. Furthermore, a digester feed material may be added to digestion system 260 to enhance the production of methane. For example, FOG may be added to digestion system 260 to be digested along with the liquid centrate. As mentioned above, solid materials recovered from digestion system 260 may be directed to DAF/CAF thickening system 250 and returned to the process. Remaining liquid may be purged from the process or further processed. Biogas generated in digestion system 260, which is rich in methane and carbon dioxide, may be used as fuel in the process, such as fuel for dryer 210.

The cake that exits centrifuge 150 may contain essentially equal amounts of solid and liquid materials. More specifically, the cake may contain as little as approximately 30%, 35%, 40%, or 45% solid materials by weight or as much as approximately 50%, 55%, 60%, 65%, or more solid materials by weight. Because the cake contains some liquid materials, some NH₃—N dissolved therein exits centrifuge 150 in the cake. The cake exiting centrifuge 150 may undergo solar drying to reduce and/or eliminate the need for a subsequent thermal drying process.

A halide may optionally be added to any stream of the process to reduce mercury emissions during combustion of the final product. According to an exemplary embodiment of the present invention, the halide may be added to the cake exiting centrifuge 150, which reduces the amount of the halide that would be lost along with the liquid centrate stream exiting centrifuge 150 if added prior to centrifuge 150. The halide may be in the form of a solid salt or a concentrated, aqueous solution, for example. The halide may contain fluoride, chloride, bromide, iodide, or a combination thereof. For example, the halide may contain NaCl, HCl, HBr, CaBr₂, or another halide. In the illustrated embodiment of FIG. 1, a concentrated, aqueous solution is provided in halide tank 160, delivered via pump 165, and sprayed onto the cake exiting centrifuge 150. Treating the cake with an aqueous halide solution may reduce mercury emissions during combustion of the final product. Even without the addition of a halide, the cake itself, and in particular the liquid portion of the cake, may contain dissolved halides. However, adding an additional quantity of halides to the cake may further reduce mercury emissions during combustion of the final product. The addition of chloride and bromide, in particular, to fossil fuels reduces mercury emissions during combustion by oxidizing mercury present in the fuel and by increasing the presence of particulate-bound mercury in the fuel. One such process is disclosed in International Publication No. WO 2005/092477, the disclosure of which is expressly incorporated herein by reference. Oxidized mercury, which is soluble in water, is easier to scrub out with conventional gas clean-up equipment than elemental mercury, which is not soluble in water. Additionally, particulate collectors used to remove particulate-bound mercury are more efficient than standard equipment used to remove vapor-phase mercury. Therefore, adding halides to the cake may oxidize mercury present in the final product and may increase the presence of particulate-bound mercury in the final product. As a result, adding halides to the cake may promote the capture of mercury released during combustion of the final product. Halide addition may be especially important for fuels produced from biosolids, because biosolids may contain more mercury than fossil fuels.

After exiting centrifuge 150, the solid cake may enter mixer 200. Mixer 200 may include any device capable of mixing the solid cake and other inputs, for example, a pugmill mixer. In mixer 200, the solid cake may be mixed with feedstock bypassed from the incoming flow from WWTP 10 and before pump 30. As mentioned above, a portion of the feedstock may be directed via bypass pump 95 to mixer 200 to take advantage of excess space in dryer 210. The solid cake may also be mixed with some dried solids from dryer 210, as described in more detail below. After the solid cake, bypassed feedstock, and dried solids are sufficiently mixed in mixer 200, the mixed solids may be conveyed to dryer 210 by a suitable mode of transport. Before even entering dryer 210, the mixed solids from mixer 200 may contain primarily solid materials due to mixing with dried solids. More specifically, the solid materials entering dryer 210 may contain as little as approximately 50%, 55%, or 60% solid materials by weight or as much as approximately 65%, 70%, or 75% solid materials by weight.

Referring still to FIG. 1, dryer 210 is provided to further dry the mixed solids from mixer 200. Dryer 210 may receive energy from various external and internal process streams. For example, dryer 210 may receive flue gas exhausted from a heat transfer system of the process. Dryer 210 may also receive natural gas, such as methane. The natural gas may be supplied to dryer 210 at a temperature of over 600° F. (316° C.). Dryer 210 may also receive recirculation gas from ammonia scrubber 300 (FIG. 3), which is provided to treat gas exhausted from the process. Dryer 210 may also receive biogas from digestion system 260, which is described in more detail above. The output from dryer 210 includes a dried solid product and vaporized materials driven off from the solid product. These outputs from dryer 210 may be directed through a separator, such as a cyclone separator, to enhance separation of the dried solid product from the vaporized materials. The vaporized materials exiting dryer 210 may be subjected to further treatment, such as the vapor treatment processes illustrated in FIG. 3 for treating liberated vapor from pressure letdown tank 100. For example, the vapors exiting dryer 210 may be directed to ammonia scrubber 300, sodium oxide scrubber 305, and/or regenerative thermal oxidizer 310.

The dried solid product may be dried in dryer 210 to yield particles of a desired size, wherein the particles are large enough to prevent dusting, yet are small enough to provide a high surface area such that the particles may be easily combusted. Each macroscopic particle may be an agglomerate of smaller, microscopic particles weakly bound together. The macroscopic particle may have a diameter as small as approximately 1 millimeter, 3 millimeters, or 5 millimeters and as large as approximately 1 centimeter, 3 centimeters, or more, for example. The density of the macroscopic particles may be as low as approximately 0.5 g/cc, 1.0 g/cc, or 1.5 g/cc and as high as approximately 2.0 g/cc, 2.5 g/cc, or 3.0 g/cc, for example. The dried solid product may contain macroscopic particles of various sizes and densities. The microscopic particles that bind together to form larger, macroscopic particles may be as small as approximately 0.1 micrometer, 1 micrometer, 3 micrometers, 5 micrometers, or 10 micrometers in size and as large as approximately 50 micrometers, 75 micrometers, 100 micrometers, 125 micrometers, or 150 micrometers in size. An exemplary size distribution of solid particles in the processed slurry is set forth in Table 1 above. The microscopic particles in the dried solid product may have an essentially similar size distribution. If the particles are too large, they may be crushed or otherwise processed to produce particles of smaller size. If the particles are too small, they may be recycled through the process and mixed with incoming solids.

The dried solid product exiting dryer 210 may contain primarily solids. More specifically, the dried solid product exiting dryer 210 may contain as little as approximately 50%, 55%, 60%, 65%, 70%, 75%, or 80% solid materials by weight or as much as approximately 85%, 90%, or 95% solid materials by weight. Therefore, the dried solid product exiting dryer 210 may have a moisture content as low as approximately 5%, 10%, or 15% by weight or as high as approximately 20%, 25%, or 30% by weight. This percentage of solid materials in the dried solid product is significantly higher than the percentage of solid materials in the incoming feedstock from WWTP 10. The volume of dried solid product exiting dryer 210 depends on the bulk density of the dried solid product. The bulk density of the dried solid product may be as low as approximately 0.5 g/cc, 0.6 g/cc, or 0.7 g/cc or as high as approximately 0.8 g/cc, 0.9 g/cc, and 1.0 g/cc, for example. The dried solid product exiting dryer 210 may be cooled, for example, in a heat exchanger by exchange with process cooling water before undergoing subsequent processing.

Referring still to FIG. 1, a portion of the dried solid product from dryer 210 may be recycled back to mixer 200 to be mixed with the solid cake from centrifuge 150. The amount of dried solid product recycled through mixer 200 and dryer 210 may be altered to control the moisture level in dryer 210 and the stickiness of the material in mixer 200 and dryer 210. For example, approximately 30%, 40%, 50%, 60%, 70%, 80%, or more by weight of the dried solid product may be recycled back to mixer 200 and dryer 210. The dried solid product that is recycled through mixer 200 may include particles that are below a threshold size. The remaining portion of the dried solid fuel product from dryer 210 may be stored and accumulated in silo 220, optionally under a nitrogen blanket. Then, the solid fuel product may be delivered to customer 235, such as via truck 230 or another suitable mode of transport.

As mentioned above, the solid fuel product contains a mixture of primarily solid materials and some remaining liquid materials. More specifically, the solid fuel product may have a moisture content as low as approximately 5%, 10%, or 15% by weight or as high as approximately 20%, 25%, 30%, 35%, 40%, 45% or 50% by weight. The solid fuel product may include small amounts of NH₃—N dissolved in the remaining liquid portion.

An experimental dry-basis proximate analysis of a sample of the solid fuel product is set forth in Table 3 below. Ignoring the moisture content, the dry-basis proximate analysis sets forth the composition of the solid fuel product in terms of volatile components (when heated to 950° C.), the fixed carbon remaining at that point, and ash. This information is set forth in Table 3 below.

TABLE 3

| | Weight Percent |
|---|---|
| Material | (Dry Basis) |
| Ash | 48.72% |
| Volatile | 46.60% |
| Fixed Carbon | 4.68% |

An experimental dry-basis ultimate analysis of a sample of the solid fuel product is set forth in Table 4 below. Ignoring the moisture content, the dry-basis ultimate analysis breaks down the composition of the volatile components and the fixed carbon in terms of carbon, hydrogen, and oxygen (the major components) as well as sulfur and nitrogen.

TABLE 4

| | Weight Percent |
|---|---|
| Material | (Dry Basis) |
| Ash | 48.72% |
| Carbon | 35.91% |
| Hydrogen | 4.65% |
| Oxygen | 6.04% |
| Sulfur | 2.02% |
| Nitrogen | 2.66% |

An experimental composition of the ash in a sample of the solid fuel product is broken down in Table 5 below. Ash includes substances, such as sand, that are received from WWTP 10 and not modified by the process described above.

TABLE 5

| Material | Weight Percent |
|---|---|
| SiO₂ | 32.60% |
| Al₂O₃ | 9.86% |
| TiO₂ | 1.53% |
| Fe₂O₃ | 18.50% |
| CaO | 9.60% |
| MgO | 3.35% |
| Na₂O | 0.79% |
| K₂O | 1.04% |
| P₂O₅ | 14.35% |
| SO₃ | 6.02% |
| Cl | 0.05% |
| CO₃ | 0.45% |

Experimental combustion parameters of a sample of the solid fuel product are set forth in Table 6 below. The higher heating value (HHV) represents the heat released from complete combustion of the solid fuel product to carbon dioxide and with condensation to liquid water. Unlike raw biosolids, the solid fuel product has a positive heating value. Therefore, it can be combusted to produce heat. The moisture and mineral free heating value (MMFHV) represents the heat that would be released were the remaining moisture to be removed from the solid fuel product. The mineral and ash free heating value (MAFHV) represents the heat that would be released were the remaining moisture and ash to be removed from the solid fuel product.

TABLE 6

| | Heat |
|---|---|
| Parameter | (BTU/lb) |
| HHV | 6503.00 |
| MMFHV | 13836.00 |
| MAFHV | 12682.00 |

Experimental ash fusion parameters of a sample of the solid fuel product are set forth in Table 7 below. In general, the ash fusion parameters indicate the softening and melting behavior of the ash in the solid fuel product. There are not distinct melting points for ash like with ice, but as the ash is heated, its viscosity decreases. Eventually, the ash becomes “slag,” a glassy, vitrified material that may be used, for example, as an aggregate for roads, roofing shingles, or other construction applications. This softening and melting behavior is measured microscopically at four defined points under oxidizing conditions. The ash particles are shaped like small cones. The initial deformation temperature (IT) represents the temperature at which a point of the cone begins to round. The softening temperature (ST), also referred to as the spherical temperature, represents the temperature at which a base of the cone is equal to its height. The hemispherical temperature (HT) represents the temperature at which the base of the cone is twice its height. The fluid temperature (FT) represents the temperature at which the cone has spread to a fused mass no more than 1.6 millimeters in height.

TABLE 7

| | Temperature |
|---|---|
| Parameter | (° F.) |
| IT | 2094 |
| ST | 2142 |
| HT | 2178 |
| FT | 2191 |

Advantageously, the solid fuel product has a high burn rate relative to other fuels, including coal. The high burn rate may be attributed to the oxygen content of the solid fuel product, the small particle size, and/or a catalytic contribution from one or more of the inorganic species present. When combusted as a fuel source, the solid fuel product releases energy over a short amount of time, resulting in a quick, hot flame. If the solid fuel product is gasified, the high burn rate may allow for smaller sized equipment.

Also advantageously, the solid fuel product is less sticky than other biosolid products, making the solid fuel product easier to process, transport, and use. Individual particles of the solid fuel product may include a coating of char carbon encapsulating tar-like materials.

Another advantage of the solid fuel product may be its natural halogen content. As discussed above, fuels release mercury when combusted. The addition of chloride and bromide, in particular, to fossil fuels reduces mercury emissions during combustion by oxidizing mercury present in the fuel and by increasing the presence of particulate-bound mercury in the fuel. Oxidized mercury, which is soluble in water, is easier to scrub out with conventional gas clean-up equipment than elemental mercury, which is not soluble in water. Additionally, particulate collectors used to remove particulate-bound mercury are more efficient than standard equipment used to remove vapor-phase mercury. As set forth in Table 5 above, natural amounts of chlorine have been measured in the solid fuel product. The solid fuel product may also contain other halogens, including fluorine, bromine, and iodine. Without even having to add halides to the process, such as in the form of a concentrated aqueous halide stream, the natural halogen content of the solid fuel product may promote mercury oxidation and the presence of particulate-bound mercury to reduce mercury emissions during combustion of the solid fuel product.

Before being delivered to customer 235, the dried solid product may be blended with other substances, including biomass. For example, the dried solid product may be mixed with low-rank coals. The addition of biomass and other substances may enhance the heating value of the final product.

While this invention has been described as having preferred designs, the present invention can be further modified within the spirit and scope of this disclosure. This application is therefore intended to cover any variations, uses, or adaptations of the invention using its general principles. Further, this application is intended to cover such departures from the present disclosure as come within known or customary practice in the art to which this invention pertains and which fall within the limits of the appended claims.

As used herein, the following terms have the following meanings. “Comprising” or “comprises” means including, without limitation. “Including” or “includes” means including, without limitation. “Or” means and/or. “A” means one or more.

What is claimed is:

1. A method comprising:
(a) providing a feedstock that contains organic material;
(b) providing a feedstock that contains organic material that that has been processed into a slurry by heating and pressurizing to a pressure that substantially inhibits water in the slurry from vaporizing;
(c) at least partially evenly mixing the feedstock of (a) and the slurry of (b); and
(d) heating the mixture of (c).

2. The method of claim 1, wherein the feedstock of (b) is pressurized prior to heating.

3. The method of claim 1, further comprising introducing the slurry of (b) to the feedstock of (a) prior to said step (c).

4. The method of claim 1, wherein step (c) occurs in one of a mechanically-driven mixing device and a static mixing device.

5. The method of claim 1, wherein said step (c) further comprises reducing the viscosity of the mixture of (c) to less than 10,000 cp.

6. The method of claim 1, wherein said step (c) further comprises raising the temperature of the mixture of (c) to at least 100° F. (38° C.).

7. The method of claim 1, wherein, subsequent said step (d), at least a portion of the mixture of (c) comprises the feedstock of (b).

8. The method of claim 1, wherein the organic material comprises at least one of sewage, sewage sludge, and biosolids.

9. A method comprising:
(a) providing a feedstock containing organic material and ammonia;
(b) pressurizing and heating the feedstock to provide a processed slurry;
(c) flashing the processed slurry to reduce the pressure and temperature of the processed slurry and to vaporize at least a portion of the ammonia; and
(d) recovering the portion of the ammonia vaporized during said step (c).

10. The method of claim 9, wherein said flashing step (c) occurs after said pressurizing step (b) and prior to said recovering step (d).

11. The method of claim 9, wherein said step (c) further comprises vaporizing water and carbon dioxide.

12. The method of claim 9, wherein said step (d) further comprises distilling the vaporized components to recover a liquid stream of water and ammonia.

13. The method of claim 9, wherein the portion of the ammonia vaporized in said step (c) comprises between 10% and 70% by weight of the ammonia in the feedstock.

14. The method of claim 9, wherein said step (c) further comprises reducing the pressure of the processed slurry to a pressure above the saturation pressure of water at the reduced temperature.

15. A method comprising:
(a) providing a feedstock containing organic material;
(b) pressurizing and heating the feedstock to provide a processed slurry;
(c) dewatering the processed slurry; and
(d) adding a halide to provide a fuel product.

16. The method of claim 15, wherein said adding step (d) occurs after said dewatering step (c).

17. The method of claim 15, wherein the halide of step (d) comprises at least one of fluoride, chloride, bromide, and iodide.

18. The method of claim 15, wherein said adding step (d) further comprises treating the processed slurry with an aqueous halide solution.

19. The method of claim 15, further comprising combusting the solid fuel product to promote mercury oxidation.

20. A method comprising:
(a) providing a feedstock that contains organic material;
(b) processing the feedstock into a slurry by heating and pressurizing the slurry to a pressure that substantially inhibits water in the slurry from vaporizing;
(c) dewatering the processed slurry to provide a liquid centrate; and
(d) digesting to produce methane at least a portion of the processed slurry after the processing of (b) and prior to the dewatering of (c).

21. The method of claim 20, further comprising digesting to produce methane the liquid centrate to provide a processed centrate.

22. The method of claim 20, further comprising combining at least a portion of the processed centrate with the processed slurry.

23. The method of claim 20, wherein the digesting of (d) occurs in the presence of a digester feed to enhance production of methane.

24. The method of claim 23, wherein the digester feed comprises at least one of a fat, an oil, or a grease.
